(19) United States
(12) Patent Application Publication
(10) Pub. No.: US 2025/0000570 A1
(43) Pub. Date: Jan. 2, 2025

(54) Radiofrequency Cannula for Thermal Ablation

(71) Applicant: Ming Lin, Irvine, CA (US)

(72) Inventor: Ming Lin, Irvine, CA (US)

(21) Appl. No.: 18/217,540

(22) Filed: Jul. 1, 2023

(51) Int. Cl. A61B 18/14

(52) U.S. Cl. CPC A61B 18/1482; A61B 2018/00083; A61B 2018/00577

(57) ABSTRACT

A radiofrequency cannula for thermal ablation is configured to have an electrode guiding arrangement built-in, without any foreign material, chemical or element involved, to guide an active electrode tip of a radiofrequency electrode inserted into an inner electrode passage of the radiofrequency cannula to turn and extend out through an electrode outlet opening formed in a side of the radiofrequency cannula. The radiofrequency electrode guiding arrangement is embodied to comprise an electrode guiding lobe formed by bending a portion of a side wall of an active tip portion into the inner electrode passage to form an inclined stopper to not only guide the active electrode tip of radiofrequency electrode to extend out through the electrode outlet opening, but also prevent any body tissue entered in the active tip portion blocking the electrode outlet opening of the radiofrequency cannula.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

The present invention relates to thermal ablation, and more particularly to a radiofrequency cannula for thermal ablation with built-in electrode guiding arrangement configured to guide an active end portion of a radiofrequency electrode which is inserted through the cannula to turn and penetrate through a radiofrequency electrode outlet opening formed in a side of the cannula precisely and smoothly without blind angle while preventing tissue embedded in the active tip portion of the cannula from blocking the electrode outlet opening as well as the inner electrode passage of the cannula.

### Description of Related Arts

Radiofrequency cannula thermal ablation is a medical procedure used to ablate tissue, where high-frequency electrical currents are used to generate heat and destroy tissue. Cannula thermal ablation may treat various conditions by selectively destroying abnormal or diseased tissue using high-radiofrequency electrical currents. Radiofrequency ablation (RFA) or radiofrequency neurectomy uses radio waves to create a current that heats a small area of nerves. The heat destroys that area of the nerve, stopping it from sending pain signals to the brain. It generally provides lasting relief for people with chronic pain, especially in the lower back, neck and arthritic joints.

A cannula, which is a thin, needle-like device with a radiofrequency electrode at the tip, is inserted into the target tissue. The cannula serves as a conduit for the passage of radiofrequency energy. In many cases, imaging techniques such as ultrasound, CT scan, or MRI are used to guide the precise placement of the cannula within the target tissue.

Once the cannula is properly positioned, radiofrequency energy is delivered through the radiofrequency electrode at the tip. The energy generates heat, which creates a thermal lesion in the target tissue. The generated heat causes coagulation and destruction of the targeted tissue. The radiofrequency energy can denature proteins and disrupt cellular structures, leading to irreversible tissue damage. The ablation process must be carefully monitored to avoid damage to adjacent healthy tissues. Once the desired tissue ablation is achieved and the radiofrequency energy is discontinued, the radiofrequency electrode is withdrawn from the cannula and then the cannula is slowly withdrawn from the tissue.

Radiofrequency cannula tissue ablation can be used for various medical applications, including the treatment of solid tumors, liver or kidney lesions, varicose veins, and chronic pain conditions like facet joint syndrome.

U.S. Pat. No. 5,213,578, as shown in FIG. 1, discloses an epidural cannula 1 having a radial outlet opening 2 provided in a side of a concave wall portion 3 in a bending zone along a center line 5 of the cannula 1. As it is necessary to pierce the skin and muscle tissue with the tip of the epidural cannula, a mandrel 6 is inserted into the epidural cannula to prevent the punching out of skin and tissue parts, the mandrel forming with a distal end piece 7 a flush closure for the radial outlet opening 2 of the epidural cannula 1. The mandrel 6 is provided with a recess 8 in the form of a bore coaxial with its longitudinal axis, the cross-section of the bore corresponding to the projection plane of the axial outlet opening 4 in the epidural cannula, and the bore as well as the latter being centered on the longitudinal center line of the epidural cannula 1. Over a partial length of the mandrel 6, the mandrel is cut away between the axial outlet opening 4 in the direction toward the proximal end, whereby the mandrel 6 is flattened over one half of its cross-section. The thinned neck portions interposed between distal end 7 and the proximal end of the mandrel is flexible and thus, upon insertion of the mandrel 6 into the cannula 1, the distal end snaps radially into the bent portion of cannula 1 releasably locking the mandrel 6 in the position.

Although the mandrel 6 also serves to guide a spinal cannula 9 through the epidural cannula 1 and through the axial outlet opening 4 thereof, to precisely and smoothly penetrate the tip portion of the epidural cannula 1 through the axial outlet opening 4 at the bending wall portion 3 is difficult and requires excellent technique and experience to accomplish.

FIG. 2 illustrates an endocardial ablation device 2, for examples U.S. Pat. Nos. 7,122,034, 7,229,438, 8,152,805, and 10,736,689, comprising a sheath 6 having a side port 62 operated as an exit port in an anchoring member 58 before a distal finger 64 thereof, wherein a curved catheter 4 having an attached ablation electrode 8 is bent and penetrates through the side port 62, wherein the side port 62 has to be long and large enough to allow the bent tip portion of the catheter 4 to penetrate therethrough that may cause tissue being embedded in the sheath 6 through the side port 4 and difficulty to extend the catheter 4 thereto.

In view of above, although the side port formed at a bent tip portion of the cannula allows the cathode to penetrate and extend out and form two spaced apart active electrode tips for current flowing through the two tips simultaneously to have a larger ablation area and cause a larger coagulation and destruction of the targeted tissue. However, the drawback of such side port of the cannula is the penetration process of the flexible cathode through the side port. Therefore, the bending angle of the curved distal tip of the cannula is limited to position and align the side port along a longitudinal axis A-A′ of the cannula, as shown in FIG. 3 and FIG. 4, so as to facilitate the cathode to straightly extending out.

A conventional solution to guide a bent cathode to extend out of the side port of a straight cannula, as illustrated in FIG. 4A, and to guide a straight cathode to extend out of the side port of a bent cannula, as illustrated in FIG. 4B, by inserting a closure member 3 into the distal tip portion 11 of the cannula 1 to fill the tubular space between the distal end and the side port 12 of the cannula 1 such that the closure member 3 blocks the cathode 4 to extend from the distal end of the cannula and the inclined surface of the closure member 3 guides the cathode 4 to extend out via the side port 12. The difficulty of reducing this type cannula to practice is how to affix the closure member 3 with the tip portion of the cannula 1 to avoid the closure member 3 being fallen off and leaving inside the human body while inserting and withdrawing of the cannula 1 to the human body. It is because the cannula 1 is a metal made needle tube as thin as 1 mm in diameter that must meet the ISO 9626 requirement and no chemical adhesive may be used to adhere the closure member 3.

### SUMMARY OF THE PRESENT INVENTION

The invention is advantageous in that it provides a radiofrequency (RF) cannula with a built-in electrode guiding arrangement for thermal ablation, which is configured to guide an active electrode tip of an inserted radiofrequency electrode to turn and penetrate through an electrode outlet opening formed in a side of the radiofrequency cannula precisely and smoothly without blind angle.

Another advantage of the invention is to provide a radiofrequency cannula with a built-in electrode guiding arrangement for thermal ablation, wherein the electrode guiding arrangement is integrally formed in the radiofrequency cannula without any foreign element required to be affixed or installed thereto nor chemical required to be used.

Another advantage of the invention is to provide a radiofrequency cannula with a built-in electrode guiding arrangement for thermal ablation, which significantly prevents any tissue embedded in an active tip portion of the radiofrequency cannula from blocking the electrode outlet opening as well as an inner electrode passage of the radiofrequency cannula.

Additional advantages and features of the invention will become apparent from the description which follows and may be realized by means of the instrumentalities and combinations particular point out in the appended claims.

According to the present invention, the foregoing and other objects and advantages are attained by a radiofrequency cannula for thermal ablation, comprising:
a radiofrequency cannula for insertion into a body tissue, comprising:
a metal made tubular cannula shaft having a bevel tip which has a distal end opening, a connection end which has a proximal end opening, an inner electrode passage extending from the proximal end opening to the distal end opening along a longitudinal axis of the cannula shaft, a curved portion between the bevel tip and the connection end to define an elongated shaft body extended along the longitudinal axis and between the curved portion and the connection end and an active tip portion extended between the curved portion and the bevel tip and defined a predetermined included angle with respect to the longitudinal axis, and a radial electrode outlet opening provided a short side of the active tip portion in form of an elongated longitudinal hole, having a proximal end and a distal end, communicating the inner electrode passage with outside;
an electrode guiding arrangement, which is provided at the electrode outlet opening of the cannula shaft, comprising an electrode guiding lobe integrally extended from the distal end of the electrode outlet opening inwardly for a predetermined angle with respect to the longitudinal axis to form a stopper between the distal end opening and the electrode outlet opening and define a guiding surface facing the inner electrode passage; and
a luer-lock connection member connected to the connection end of the cannula shaft; and
a mandrel configured to be inserted into the inner electrode passage of the radiofrequency cannula and extended from the proximate end opening to the electrode guiding lobe while inserting the radiofrequency cannula in the body tissue and to be withdrawn from the inner electrode passage of the radiofrequency cannula after the active tip portion of the radiofrequency cannula reaching a desired tissue part;
thereby the radiofrequency cannula is adapted to equipped with a radiofrequency electrode for thermal ablation, wherein the radiofrequency electrode has an active electrode tip and an outer diameter smaller than a size of the inner electrode passage and the electrode outlet opening, configured to insertion within the inner electrode passage of the radiofrequency cannula inserted in the body tissue after the mandrel is withdrawn from the inner electrode passage, wherein the active electrode tip is guided by the electrode guiding lobe to penetrate out of the inner electrode passage through the electrode outlet opening such that the active electrode tip of the radiofrequency electrode and the active tip portion of the radiofrequency cannula have a predetermined distance and define a predetermined included angle therebetween.

In one embodiment, the radiofrequency cannula further comprises an insulation covering and extending between the connection end and the curved portion of the cannula shaft.

In one embodiment, the radiofrequency cannula has an outside cross-sectional dimension consistent with its intended use, typically being 0.5 mm to 5 mm, usually form 1 mm to 4 mm and an inner cross-sectional dimension in the range from 0.3 mm to 4 mm, preferably from 0.5 mm to 3.5 mm. The cannula can also have other outside and inner cross-sectional dimensions in other embodiments.

In one embodiment, the electrode guiding lobe is integrally formed by bending a portion of a cannula side wall at the active tip portion of the cannula shaft inwardly to inclinedly extend in the inner electrode passage until a lobe end of the electrode guiding lobe in contact with an opposing inner surface of the inner electrode passage.

In accordance with another aspect of the invention, the present invention provides a producing method of an electrode guiding arrangement of a radiofrequency cannula, comprising steps of:
(a) forming an elongated longitudinal hole in form of U-shape in a short side of an active tip portion of an elongated and tubular cannula shaft of the radiofrequency cannula and an electrode guiding lobe having two lobe sides and a lobe end, wherein the two lobe sides and the lobe end are surrounded by the U shape hole;
(b) bending the electrode guiding lobe into an inner electrode passage of the radiofrequency cannula about a root end thereof until the lobe end is in contact with an opposing inner surface of the inner electrode passage, such that the elongated longitudinal hole forms a radial electrode outlet opening of the radiofrequency cannula to communicate the inner electrode passage with outside and the electrode guiding lobe forms an inclined stopper for a distal end opening of the radiofrequency cannula to prevent tissue entered through the distal end opening from blocking the electrode outlet opening and entering a portion of the inner electrode passage extending between the electrode guiding lobe and a proximal end opening of the inner electrode passage; and
(c) forming a curved portion between the electrode outlet opening and the proximal end opening of the cannula shaft to define an active tip portion, extending between the distal end opening and the curved portion, which has a predetermined slant angle with respect to a longitudinal axis of the cannula shaft.

Still further objects and advantages will become apparent from consideration of the ensuing description and drawings. These and other objectives, features, and advantages of the present invention will become apparent from the following detailed description, the accompanying drawings, and the appended claims.

### BRIEF DESCRIPTION OF THE DRAWINGS

FIG. 1 is schematic view illustrating a conventional epidural cannula.

FIG. 2 is a perspective view illustrating a conventional endocardial ablation device.

FIG. 3 is a schematic view illustrating a conventional radiofrequency cannula with a bent tip portion and a side port.

FIG. 4A and FIG. 4B are sectional views illustrating a conventional radiofrequency cannula with a closure member inserted.

FIG. 5 is a front view of a radiofrequency cannula with built-in electrode guiding arrangement according to a preferred embodiment of the present invention.

FIG. 6 is a partial schematic view of the radiofrequency cannula according to the above preferred embodiment of the present invention.

FIG. 7 is a partial sectional view illustrating the active tip portion of the radiofrequency cannula with built-in electrode guiding arrangement according to the above preferred embodiment of the present invention.

FIG. 8 is a partial sectional view illustrating the active tip portion of the cannula shaft and the mandrel of the radiofrequency cannula according to the above preferred embodiment of the present invention.

FIG. 9 is a partial sectional view illustrating the radiofrequency cannula and the radiofrequency electrode inserted therethrough to form the radiofrequency cannula assembly according to the above preferred embodiment of the present invention.

FIG. 10 is a schematic view illustrating the active electrode tip of the radiofrequency electrode extended out through the electrode outlet opening of the radiofrequency cannula according to the above preferred embodiment of the present invention.

FIG. 11 is a front view illustrating dimensions of the active tip portion of the radiofrequency cannula before being curved according to the above preferred embodiment of the present invention.

FIG. 12 is a sectional view illustrating the active tip portion of the radiofrequency cannula before being curved according to the above preferred embodiment of the present invention.

FIG. 13 is an enlarged schematic view illustrating the electrode outlet opening and the electrode guiding lobe of the radiofrequency cannula active tip portion of the radiofrequency cannula before being curved according to the above preferred embodiment of the present invention.

FIG. 14 is an enlarged schematic view illustrating the electrode guiding lobe and the mandrel of the radiofrequency cannula assembly active tip portion of the radiofrequency cannula before being curved according to the above preferred embodiment of the present invention.

FIG. 15 is a perspective view of the radiofrequency cannula inserted with the mandrel and covered with a tubular cover shell according to the above preferred embodiment of the present invention.

FIG. 16 a perspective view of the radiofrequency cannula inserted with the mandrel according to the above preferred embodiment of the present invention.

FIG. 17 an exploded perspective view of the radiofrequency cannula and the mandrel according to the above preferred embodiment of the present invention.

FIG. 18 an exploded perspective view of the radiofrequency cannula and the radiofrequency electrode according to the above preferred embodiment of the present invention.

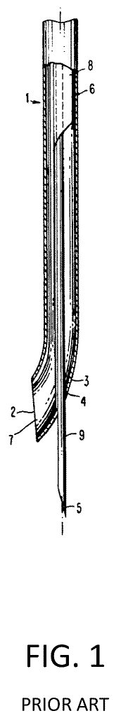

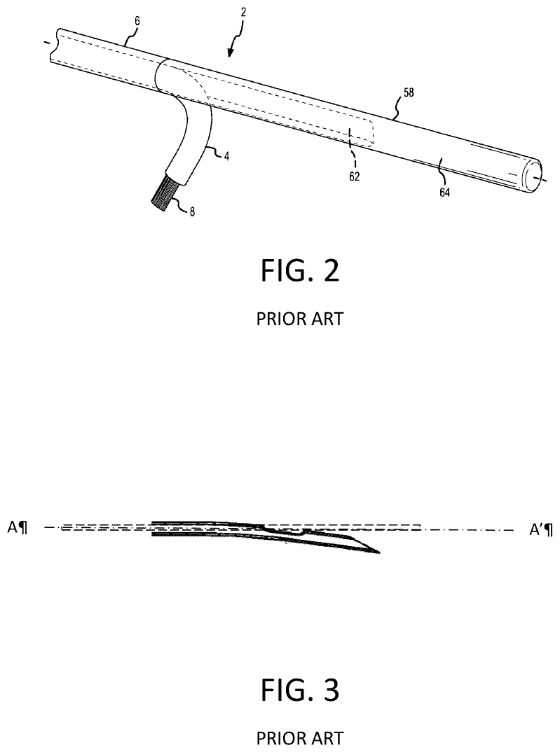

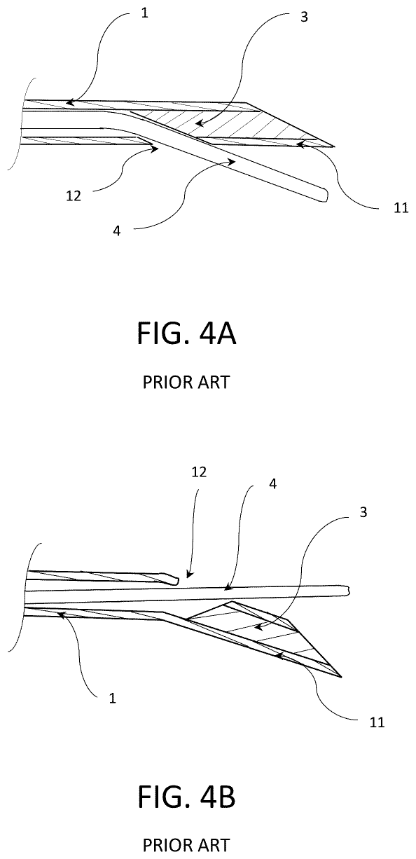

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

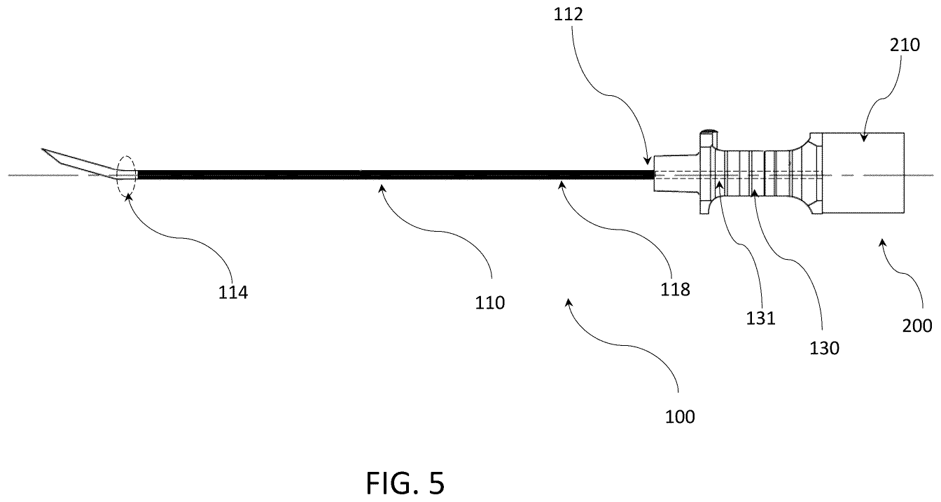

Referring to FIG. 5 to FIG. 10, a radiofrequency cannula 100 configured to be inserted into body tissue for thermal ablation according to a preferred embodiment of the present invention is illustrated. The radiofrequency cannula 100 is configured to be equipped with a mandrel 200 and a radiofrequency electrode 300 to form a radiofrequency cannula assembly.

The radiofrequency cannula 100 comprises a metal made tubular cannula shaft 110, an electrode guiding arrangement 120 and a luer-lock connection member 130.

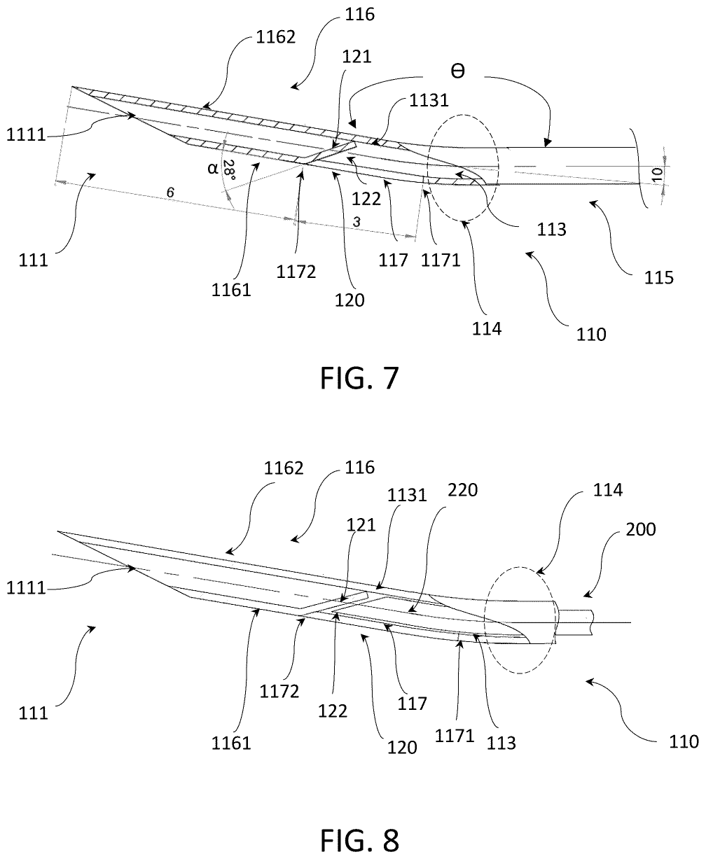

The cannula shaft 110 is made of a straight metal tube and has a bevel tip 111 having a distal end opening 1111, a connection end 112 having a proximal end opening 1121, an inner electrode passage 113 extending from the proximal end opening 1121 to the distal end opening 1111 along a longitudinal axis A-A′ of the cannula shaft 110, a curved portion 114 between the bevel tip 111 and the connection end 112 to define an elongated shaft body 115 extended along the longitudinal axis A-A′ and between the curved portion 114 and the connection end 112 and an active tip portion 116 extended between the curved portion 114 and the bevel tip 111 and defined a predetermined included angle θ (as shown in FIG. 7) with respect to shaft body 115, i.e. the longitudinal axis A-A′, and a radial electrode outlet opening 117 provided a short side 1161 of the active tip portion 116 in form of an elongated longitudinal hole, having a proximal end 1171 and a distal end 1172, communicating the inner electrode passage 113 with outside. In other words, the cannula shaft 110 is bent at the curved portion 114 to define the slant active tip portion 116 with respect to the straight shaft body 115.

The bevel tip 111 is in the form of slant surface extending from the short side 1161 to a long side 1162 of the active tip portion 116. The curved portion 114 is a portion of the cannula shaft 110 where the long side 1162 of the active tip portion 116 bends for the predetermined included angle θ towards the shaft body 115, as shown in FIG. 7, such that the electrode outlet opening 117 is formed in the short side 1161 of the active tip portion 116 adjacent to the curved portion 114.

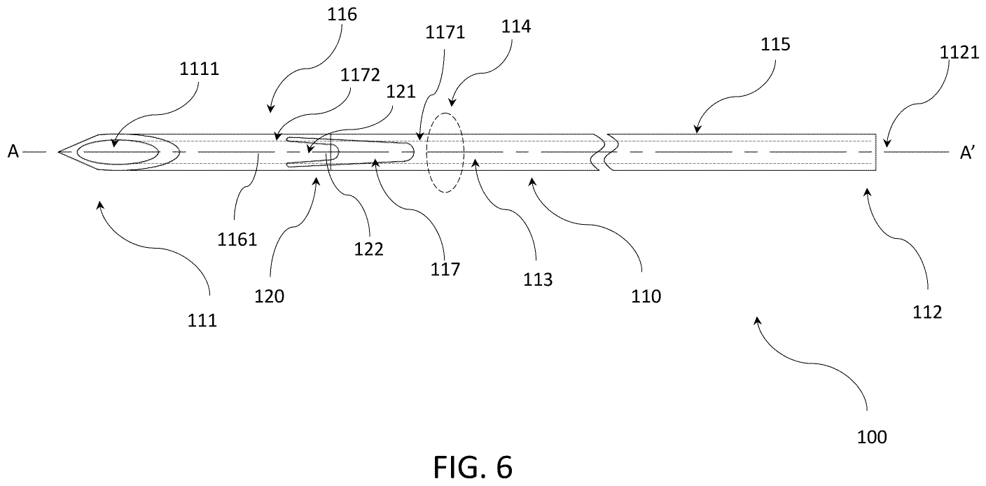

Referring to FIG. 6 to FIG. 8, the electrode guiding arrangement 120, which is provided at the electrode outlet opening 117 of the cannula shaft 110, comprises an electrode guiding lobe 121 integrally extended from the distal end 1172 of the electrode outlet opening 117 inwardly for a predetermined angle α with respect to the shaft body 115 and the longitudinal axis A-A′ to form a stopper between the distal end opening 1111 and the electrode outlet opening 117 and define a guiding surface 122 facing the inner electrode passage 113. A length of the electrode guiding lobe 121 is larger than an inner diameter of the inner electrode passage 113 so that the electrode guiding lobe 121 is inclinedly bent inwardly until a free proximal end of the electrode guiding lobe reaches an opposing inner surface 1131 of the inner electrode passage 113.

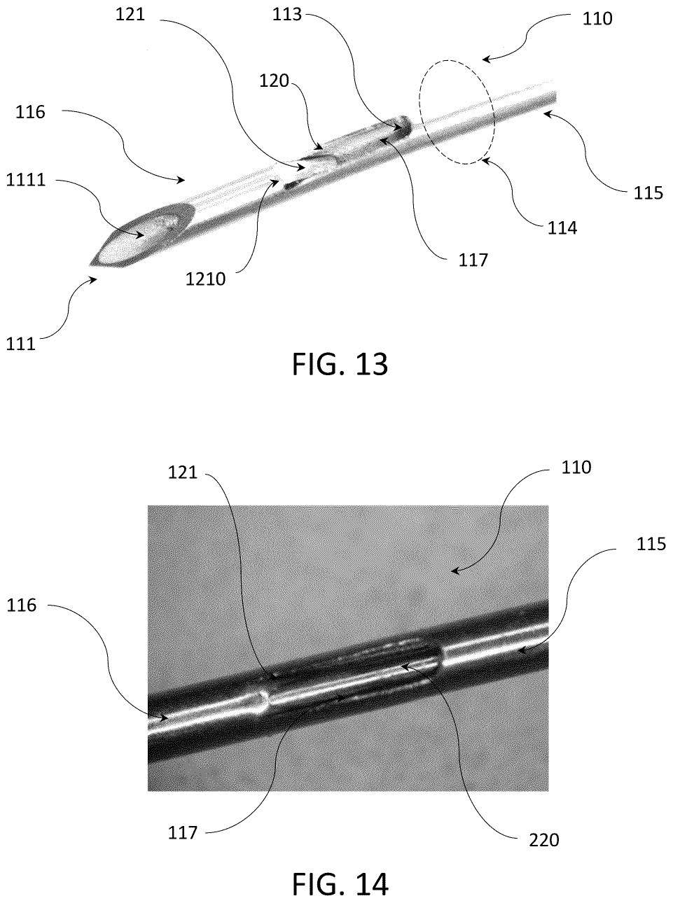

According to the preferred embodiment of the present invention, referring to FIGS. 7, 8 and 13, the electrode guiding lobe 121 is integrally formed by bending a portion of a cannula side wall at the active tip portion 116 of the cannula shaft 110 inwardly to inclinedly extend in the inner electrode passage 113 until the free proximal end of the electrode guiding lobe 121 in contact with the opposing inner surface 1131 of the inner electrode passage 113. The electrode guiding lobe 121 can gradually reduce a width thereof to its free proximal end to facilitate the bending of the electrode guiding lobe 121 about its distal end.

Preferably, the radiofrequency cannula 100 further comprises an insulation 118 covering and extending from the connection end 112 to the cured portion 114 of the cannula shaft 110, as shown in FIG. 5.

According to the preferred embodiment of the present invention, the cannula shaft 110 has an outside cross-sectional dimension consistent with its intended use, typically being 0.5 mm to 5 mm, usually form 1 mm to 4 mm and an inner cross-sectional dimension in the range from 0.3 mm to 4 mm, preferably from 0.5 mm to 3.5 mm. The cannula shaft 110 can also have other outside and inner cross-sectional dimensions in other embodiments. Referring to FIG. 7, a length of the active tip portion 114 is preferably embodied as 9 mm and a length of the electrode outlet opening 117 is 3 mm. The predetermined angle α is 28° preferably between the short side 1161 and the electrode guiding surface 122 of the inclined electrode guiding lobe 121.

The luer-lock connection member 130 is connected to the connection end 112 of the cannula shaft 110 and has an axial through hole 1301 extending to the inner electrode passage 113 of the cannula shaft 110 through its proximal end opening 1121, as shown in FIG. 5.

The mandrel 200 comprises a mandrel head 210 configured to detachably mount on the luer-lock connection member 130 and a mandrel body 220 in the form of an elongated pin coaxially extended from mandrel head 210 and inserted into the inner electrode passage 113 of the radiofrequency cannula 100 and extended from the proximate end opening 1121 to the electrode guiding lobe 121, as shown in FIG. 8 and FIG. 14, while inserting the radiofrequency cannula 100 in the body tissue and to be withdrawn from the inner electrode passage 113 of the radiofrequency cannula 100 after the active tip portion 116 of the radiofrequency cannula 100 reaching a desired tissue part.

The mandrel 200 placed in the radiofrequency cannula 100 serves not only to close the electrode outlet opening 117 to prevent tissue embedded in the electrode outlet opening 117, but moreover to strengthen the cannula shaft 110 while inserting and penetrating the body tissue.

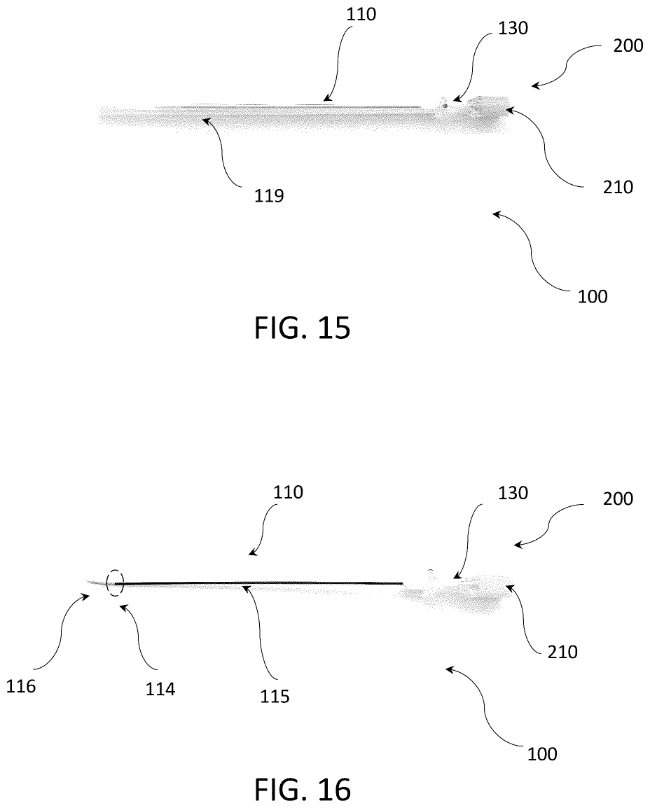

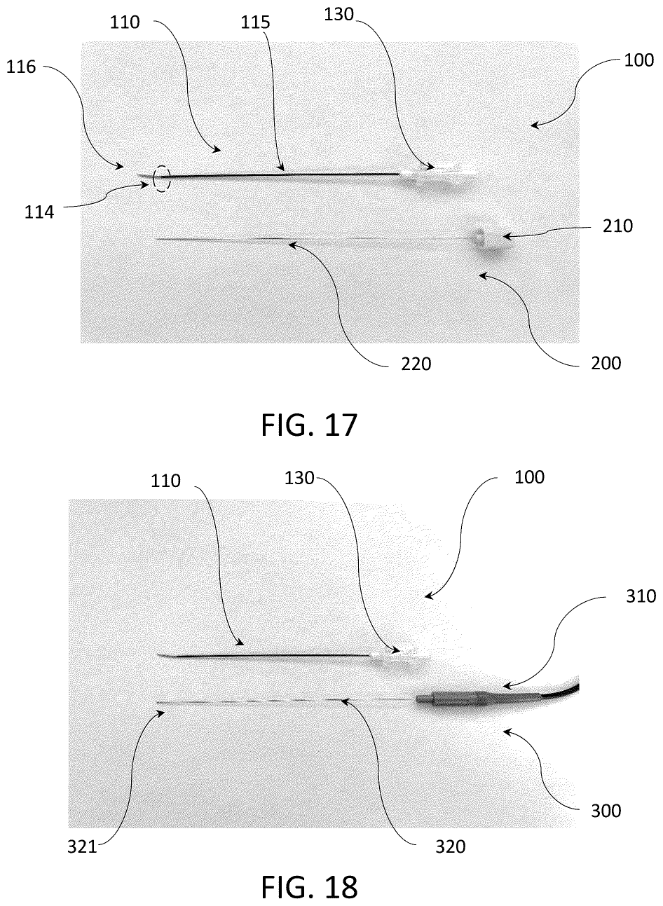

FIG. 15 to FIG. 17 illustrate images of the radiofrequency cannula 100 and the mandrel 200 and FIG. 18 illustrates the radiofrequency electrode 300 adapted to be used with the radiofrequency cannula 100 to form the tissue ablation cannula assembly. The radiofrequency cannula 100 further comprises a tubular cover shell 119 configured to cover the cannula shaft 110 after sterilized during storage and transportation before use, as shown in FIG. 15. The mandrel 200 is generally inserted into the cannula shaft 110 of the radiofrequency cannula 100 as shown in FIG. 15 and FIG. 16. FIG. 17 illustrates the mandrel 200 withdrawn from the radiofrequency cannula 100, wherein when the mandrel body 220 is inserted into the cannula shaft 110 as shown in FIG. 16, the mandrel head 210 is engaged with the luer-lock connection member 130. After the active tip portion 116 is inserted into the body tissue and reaches the desired tissue part, the mandrel 200 is pulled out from the radiofrequency cannula 100 and thus the radiofrequency electrode 300, as shown in FIG. 18, is capable of inserting into the radiofrequency cannula 100.

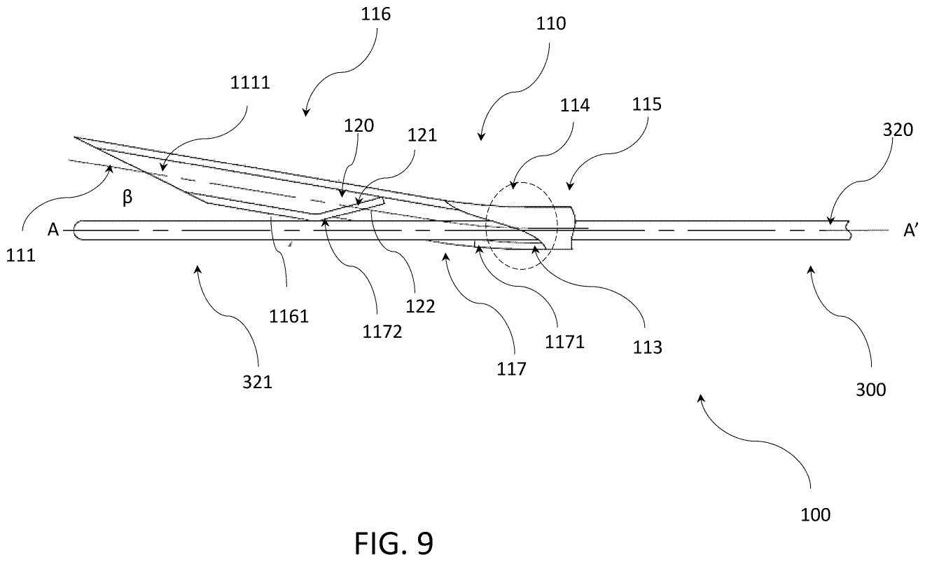

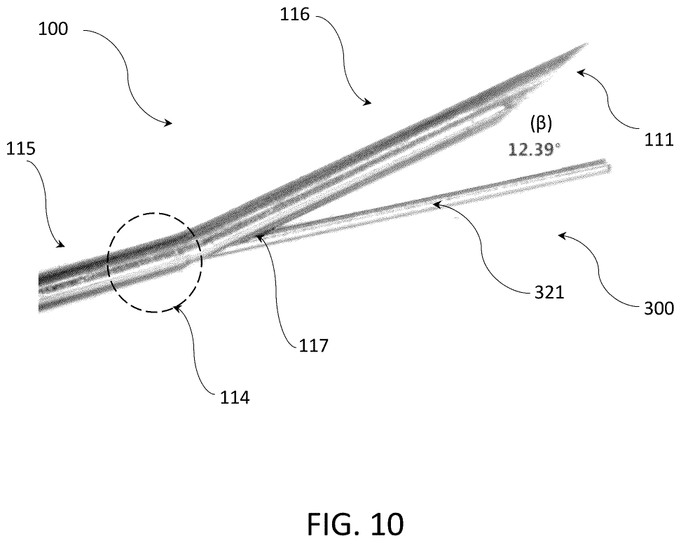

Referring to FIG. 18, the radiofrequency electrode 300 can be any type of electrode configured for thermal ablation and designed for insertion within the inner electrode passage 113 of the radiofrequency cannula 100. The radiofrequency electrode 300 comprises an electrode head 310 electrically connected to thermocouple temperature sensor, radiofrequency generator and/or electrosurgical generator and an electrode shaft 320, wherein the diameter of the inner electrode passage 113 is sized to accommodate an outer diameter of the radiofrequency electrode 300. Further, referring to FIG. 9 and FIG. 10, the electrode shaft 320 is in form of elongated pin made of electrical conducting material and extended from the electrode head 310 for a length generally equal to the length of the cannula shaft 110. A distal tip portion of the electrode shaft 320 forms an active electrode tip 321 for tissue-piecing having a length generally equal to the active tip portion 116 of the radiofrequency cannula 100. An outer diameter of the electrode body 320 is smaller than a size of the inner electrode passage 113 and the electrode outlet opening 117, such that the radiofrequency electrode 300 is adapted for insertion within the inner electrode passage 113 of the radiofrequency cannula 100 inserted in the body tissue after the mandrel 200 is withdrawn from the inner electrode passage 113, wherein the active electrode tip 321 is guided by the electrode guiding lobe 121 to penetrate out of the inner electrode passage 113 through the electrode outlet opening 117 such that the active electrode tip 321 of the radiofrequency electrode 300 and the active tip portion 116 of the radiofrequency cannula 100 have a predetermined distance and define a predetermined angle β therebetween. As an example in the preferred embodiment of the present invention, the predetermined included angle is 12.39°, as shown in FIG. 10.

It is well known that the radiofrequency (RF) generators and electrodes are able to be applied to body tissue, including but not limited to brain, spine, liver, lung, bone, vertebral bone, kidney, abdominal structures, nerves, and etc., for pain relief and functional modification such as to treat cancer and other diseases. The side extension active electrode tip 321 extended from the side hole, i.e. the electrode outlet opening 117, is uninsulated and active for applying energy, such as radiofrequency electrical energy to body tissue of a living body for the purpose of radiofrequency ablation. The active electrode tip 321 is introduced into the body tissue and to which the radiofrequency electrode 300 energizes and conducts radiofrequency current. The RF generator or electrosurgical generator connected to the radiofrequency electrode 300 applies current between the active electrode tip 321 of the radiofrequency electrode 300 and the active tip portion 116 of the radiofrequency cannula 100. Therefore, the distance between the active electrode tip 321 of the radiofrequency electrode 300 and bevel tip 111 of the active tip portion 116 of the radiofrequency cannula 100 determines the area and efficiency of each radiofrequency ablation.

The goal of radiofrequency ablation is to induce thermal injury to the tissue through electromagnetic energy deposition. The term radiofrequency refers not to the emitted wave but rather to the alternating electric current that oscillates in this frequency range. Because of the relatively high electrical resistance of tissue in comparison with the metal electrodes, there is marked agitation of the ions present in the target tissue that surrounds the electrode, since the tissue ions attempt to follow the changes in direction of the alternating electric current. The agitation results in frictional heat around the electrode. The discrepancy between the small surface area of the needle electrode and the large area of the ground pads causes the generated heat to be focused and concentrated around the needle electrode. Therefore, a control of the distance between active electrode tip 116 and the active tip portion 116 significantly controls the ablation area and efficiency.

It is appreciated that based on the formation and configuration of the electrode guiding arrangement 120 and the incorporation of the electrode guiding arrangement 120 with the electrode outlet opening 117 which is particularly formed in the active tip portion 116 of the radiofrequency cannula 100, the distance between the active electrode tip 321 of the radiofrequency electrode 300 and the bevel tip 111 of the active tip portion 116 of the radiofrequency cannula 100 can be controlled and designed by the predetermined included angle θ, the location of the electrode outlet opening 117 in the active tip portion 116 of the radiofrequency cannula 100, the length of the active tip portion 116 of the radiofrequency cannula 100, and the shape of the active electrode tip 321 of the radiofrequency electrode 300. The smaller the predetermined included angle θ or the longer the active tip portion 116 of the radiofrequency cannula 100, the longer the distance between the active electrode tip 321 of the radiofrequency electrode 300 and bevel tip 111 of the active tip portion 116 of the radiofrequency cannula 100. Also, an inward curvature or an outward curvature of the active electrode tip 321 of the radiofrequency electrode 300 respectively decreases or increases the distance between the active electrode tip 321 of the radiofrequency electrode 300 and bevel tip 111 of the active tip portion 116 of the radiofrequency cannula 100.

In addition, taking advantage of the guiding and blocking purposes of the electrode guiding lobe, the electrode outlet opening 117 may be positioned as close to the curved portion 114 as possible that can significantly maximize the distance between the bevel tip 111 and a tip end of the active electrode tip 321 with a relatively larger included angle θ, i.e. less bending of the active tip portion 116 with respect to the shaft body 115, to facilitate the penetrating of the active tip portion 116 of the radiofrequency cannula 100 in the body tissue.

Unlike the conventional side opening or side port must be provided coaxial with the longitudinal axis A-A′ in order allow the electrode shaft to penetrate through. The inclined electrode guiding lobe 121 of electrode guiding arrangement 120 serves a guiding wall to guide the active electrode tip 321, no matter it is a straight tip or curved tip, to move to the adjacent electrode outlet opening 117 and extend out of the electrode outlet opening 117. In other words, a straight electrode shaft 320 or an electrode shaft 320 having a curved active electrode tip 321 will be guided by the electrode guiding lobe 121 to extend out through the electrode outlet opening 121, or a flexible electrode shaft 320 which active electrode tip would be guided by the electrode guiding lobe 121 to curvedly extend out of the electrode outlet opening 117 too.

According to the preferred embodiment, the formation of the electrode guiding arrangement 120 is relatively easy, precise and efficient through the following steps.

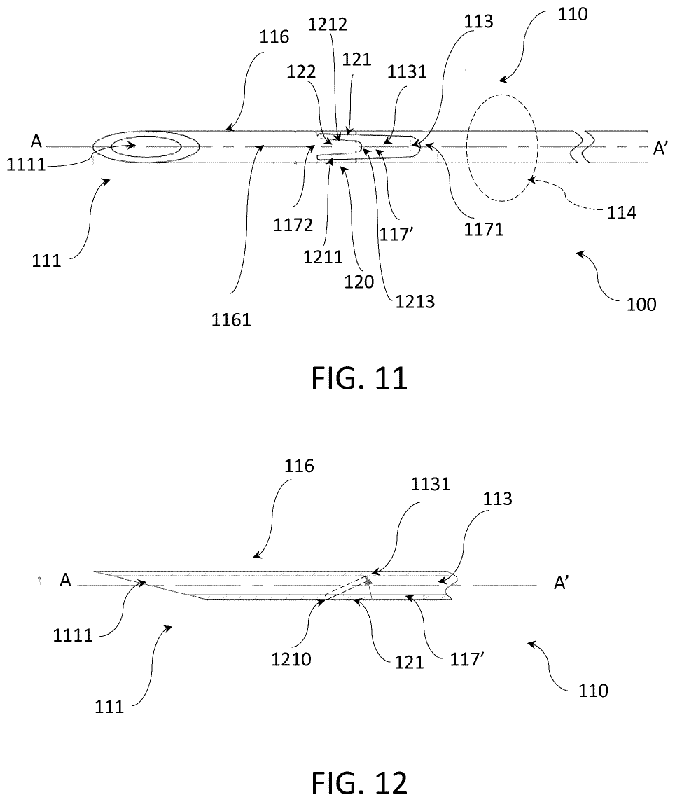

Referring to FIG. 11 and FIG. 12, firstly, an elongated and tubular metal tube is cut to produce the cannula shaft 110 with the bevel tip 111 at a distal end, wherein an elongated longitudinal hole 117′ in form of U-shape is formed in the short side 1161 of the active tip portion 116 of the cannula shaft 110 of the radiofrequency cannula 100, wherein the electrode guiding lobe 121 is defined to have two lobe sides 1211, 1212 and a lobe end 1213, wherein the two lobe sides 1211, 1212 and the lobe end 1213 are surrounded by the U shape hole 117′, as shown in FIG. 11.

Then, the electrode guiding lobe 121 is bent into the inner electrode passage 113 of the radiofrequency cannula about a root end 1210 thereof until the lobe end 1213 in contact with the opposing inner surface 1131 of the inner electrode passage 113 (as illustrated in dotted line in FIG. 12), such that the elongated longitudinal hole 117′ forms the radial electrode outlet opening 117 of the radiofrequency cannula 100 as shown in FIG. 6 to communicate the inner electrode passage 113 with outside and the electrode guiding lobe 121 forms an inclined stopper for the distal end opening 1111 of the radiofrequency cannula 100 to prevent tissue entered through the distal end opening 1111 from blocking the electrode outlet opening 117 and entering the portion of the inner electrode passage 113 extending between the electrode guiding lobe 121 and the proximal end opening 1121 of the inner electrode passage 113.

Finally, the curved portion 114 is formed between the electrode outlet opening 117 and the proximal end opening 1121 of the cannula shaft 110 to define the active tip portion 116, extending between the distal end opening 111 and the curved portion 114, which has the predetermined slant angle α with respect to the longitudinal axis A-A′ of the cannula shaft 110.

According to the present invention, the electrode guiding arrangement 120 is built-in the radiofrequency cannula 100 with the same material and body of the cannula shaft 110, so that no foreign material, chemical and/or element is involved in the configuration. The arrangement of the electrode guiding lobe 121 of the electrode guiding arrangement 120 not only substantially and efficiently guides the active electrode tip 321 of the radiofrequency electrode 300 to turn and penetrate through the electrode outlet opening 117 precisely and smoothly without blind angle, but also prevents any tissue embedded in the active tip portion 116 from blocking the electrode outlet opening 117 as well as the inner electrode passage 113 of the radiofrequency cannula 100.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A radiofrequency cannula assembly, comprising:
a radiofrequency cannula for insertion into a body tissue, comprising:
a metal made tubular cannula shaft having:
a bevel tip which has a distal end opening,
a connection end which has a proximal end opening,
an inner electrode passage extending from said proximal end opening to said distal end opening along a longitudinal axis of said cannula shaft,
a curved portion between said bevel tip and said connection end to define an elongated shaft body extended along said longitudinal axis and between said curved portion and said connection end, and an active tip portion extended between said curved portion and said bevel tip and defined a predetermined included angle with respect to said longitudinal axis, and
an electrode outlet opening provided a short side of said active tip portion in form of an elongated longitudinal hole, having a proximal end and a distal end, communicating said inner electrode passage with outside;
an electrode guiding arrangement, which is provided at said electrode outlet opening of said cannula shaft, comprising an electrode guiding lobe integrally extended from said distal end of said electrode outlet opening inwardly for a predetermined slant angle with respect to said longitudinal axis to form a stopper between said distal end opening and said electrode outlet opening and define a guiding surface facing said inner electrode passage; and
a luer-lock connection member connected to said connection end of said cannula shaft; and
a mandrel configured to be inserted into said inner electrode passage of said radiofrequency cannula and extended from said proximate end opening to said electrode guiding lobe while inserting said radiofrequency cannula in the body tissue and to be withdrawn from said inner electrode passage of said radiofrequency cannula after said active tip portion of said radiofrequency cannula reaching a desired tissue part;
thereby said radiofrequency cannula is adapted to be equipped with a radiofrequency electrode, which has an active electrode tip and an outer diameter smaller than a diameter of said inner electrode passage and a size of said electrode outlet opening, and is configured to be inserted in said inner electrode passage of said radiofrequency cannula inserted in the body tissue after said mandrel is withdrawn from said inner electrode passage, wherein said active electrode tip is guided by said electrode guiding lobe to penetrate out of said inner electrode passage through said electrode outlet opening such that said active electrode tip of said radiofrequency electrode and said active tip portion of said radiofrequency cannula have a predetermined distance therebetween and define a predetermined angle therebetween.

2. The radiofrequency cannula assembly, as recited in claim 1, wherein said electrode guiding lobe is integrally formed by bending a portion of a cannula side wall at said active tip portion of said cannula shaft inwardly to inclinedly extend in said inner electrode passage until a lobe end of said electrode guiding lobe in contact with an opposing inner surface of said inner electrode passage.

3. The radiofrequency cannula assembly, as recited in claim 1, wherein said radiofrequency cannula further comprises an insulation covering and extending between said connection end and said curved portion of said cannula shaft.

4. The radiofrequency cannula assembly, as recited in claim 2, wherein said radiofrequency cannula further comprises an insulation covering and extending between said connection end and said curved portion of said cannula shaft.

5. The radiofrequency cannula assembly, as recited in claim 1, wherein said electrode guiding lobe is formed by:
forming an elongated longitudinal hole in form of U-shape in said short side of said active tip portion of said cannula shaft of said radiofrequency cannula and said electrode guiding lobe which is defined to have two lobe sides and a lobe end, wherein said two lobe sides and said lobe end are surrounded by said U shape hole;
bending said electrode guiding lobe into said inner electrode passage of said radiofrequency cannula about a root end thereof until said lobe end is in contact with an opposing inner surface of said inner electrode passage, such that said elongated longitudinal hole forms said electrode outlet opening of said radiofrequency cannula to communicate said inner electrode passage with outside and said electrode guiding lobe forms an inclined stopper for said distal end opening of said radiofrequency cannula to prevent any tissue entered through said distal end opening from blocking said electrode outlet opening and entering a portion of said inner electrode passage extending between said electrode guiding lobe and said proximal end opening of said inner electrode passage; and
forming said curved portion between said electrode outlet opening and said proximal end opening of said cannula shaft to define said active tip portion, extending between said distal end opening and said curved portion, which has said predetermined slant angle with respect to said longitudinal axis of said cannula shaft.

6. The radiofrequency cannula assembly, as recited in claim 2, wherein said electrode guiding lobe is formed by:
forming an elongated longitudinal hole in form of U-shape in said short side of said active tip portion of said cannula shaft of said radiofrequency cannula and said electrode guiding lobe which is defined to have two lobe sides and a lobe end, wherein said two lobe sides and said lobe end are surrounded by said U shape hole;
bending said electrode guiding lobe into said inner electrode passage of said radiofrequency cannula about a root end thereof until said lobe end is in contact with said opposing inner surface of said inner electrode passage, such that said elongated longitudinal hole forms said electrode outlet opening of said radiofrequency cannula to communicate said inner electrode passage with outside and said electrode guiding lobe forms an inclined stopper for said distal end opening of said radiofrequency cannula to prevent any tissue entered through said distal end opening from blocking said electrode outlet opening and entering a portion of said inner electrode passage extending between said electrode guiding lobe and said proximal end opening of said inner electrode passage; and
forming said curved portion between said electrode outlet opening and said proximal end opening of said cannula shaft to define said active tip portion, extending between said distal end opening and said curved portion, which has said predetermined slant angle with respect to said longitudinal axis of said cannula shaft.

7. The radiofrequency cannula assembly, as recited in claim 3, wherein said electrode guiding lobe is formed by:
forming an elongated longitudinal hole in form of U-shape in said short side of said active tip portion of said cannula shaft of said radiofrequency cannula and said electrode guiding lobe which is defined to have two lobe sides and a lobe end, wherein said two lobe sides and said lobe end are surrounded by said U shape hole;
bending said electrode guiding lobe into said inner electrode passage of said radiofrequency cannula about a root end thereof until said lobe end is in contact with an opposing inner surface of said inner electrode passage, such that said elongated longitudinal hole forms said electrode outlet opening of said radiofrequency cannula to communicate said inner electrode passage with outside and said electrode guiding lobe forms an inclined stopper for said distal end opening of said radiofrequency cannula to prevent any tissue entered through said distal end opening from blocking said electrode outlet opening and entering a portion of said inner electrode passage extending between said electrode guiding lobe and said proximal end opening of said inner electrode passage; and
forming said curved portion between said electrode outlet opening and said proximal end opening of said cannula shaft to define said active tip portion, extending between said distal end opening and said curved portion, which has said predetermined slant angle with respect to said longitudinal axis of said cannula shaft.

8. An cannula for thermal ablation, comprising:
a metal made tubular cannula shaft having:
a bevel tip which has a distal end opening,
a connection end which has a proximal end opening,
an inner electrode passage extending from said proximal end opening to said distal end opening along a longitudinal axis of said cannula shaft,
a curved portion between said bevel tip and said connection end to define an elongated shaft body extended along said longitudinal axis and between said curved portion and said connection end, and an active tip portion extended between said curved portion and said bevel tip and defined a predetermined included angle with respect to said longitudinal axis, and
an electrode outlet opening provided a short side of said active tip portion in form of an elongated longitudinal hole, having a proximal end and a distal end, communicating said inner electrode passage with outside;
an electrode guiding arrangement, which is provided at said electrode outlet opening of said cannula shaft, comprising an electrode guiding lobe integrally extended from said distal end of said electrode outlet opening inwardly for a predetermined slant angle with respect to said longitudinal axis to form a stopper between said distal end opening and said electrode outlet opening and define a guiding surface facing said inner electrode passage; and
a luer-lock connection member connected to said connection end of said cannula shaft.

9. The cannula for thermal ablation, as recited in claim 8, wherein said electrode guiding lobe is integrally formed by bending a portion of a cannula side wall at said active tip portion of said cannula shaft inwardly to inclinedly extend in said inner electrode passage until a lobe end of said electrode guiding lobe in contact with an opposing inner surface of said inner electrode passage.

10. The cannula for thermal ablation, as recited in claim 8, wherein said cannula further comprises an insulation covering and extending between said connection end and said curved portion of said cannula shaft.

11. The cannula for thermal ablation, as recited in claim 9, wherein said cannula further comprises an insulation covering and extending between said connection end and said curved portion of said cannula shaft.

12. The cannula for thermal ablation, as recited in claim 8, wherein said electrode guiding lobe is formed by:
forming an elongated longitudinal hole in form of U-shape in said short side of said active tip portion of said cannula shaft of said cannula and said electrode guiding lobe which is defined to have two lobe sides and a lobe end, wherein said two lobe sides and said lobe end are surrounded by said U shape hole;
bending said electrode guiding lobe into said inner electrode passage of said cannula about a root end thereof until said lobe end is in contact with an opposing inner surface of said inner electrode passage, such that said elongated longitudinal hole forms said electrode outlet opening of said cannula to communicate said inner electrode passage with outside and said electrode guiding lobe forms an inclined stopper for said distal end opening of said cannula to prevent any tissue entered through said distal end opening from blocking said electrode outlet opening and entering a portion of said inner electrode passage extending between said electrode guiding lobe and said proximal end opening of said inner electrode passage; and
forming said curved portion between said electrode outlet opening and said proximal end opening of said cannula shaft to define said active tip portion, extending between said distal end opening and said curved portion, which has said predetermined slant angle with respect to said longitudinal axis of said cannula shaft.

13. The cannula for thermal ablation, as recited in claim 9, wherein said electrode guiding lobe is formed by:
forming an elongated longitudinal hole in form of U-shape in said short side of said active tip portion of said cannula shaft of said radiofrequency cannula and said electrode guiding lobe which is defined to have two lobe sides and a lobe end, wherein said two lobe sides and said lobe end are surrounded by said U shape hole;
bending said electrode guiding lobe into said inner electrode passage of said radiofrequency cannula about a root end thereof until said lobe end is in contact with said opposing inner surface of said inner electrode passage, such that said elongated longitudinal hole forms said electrode outlet opening of said radiofrequency cannula to communicate said inner electrode passage with outside and said electrode guiding lobe forms an inclined stopper for said distal end opening of said radiofrequency cannula to prevent any tissue entered through said distal end opening from blocking said electrode outlet opening and entering a portion of said inner electrode passage extending between said electrode guiding lobe and said proximal end opening of said inner electrode passage; and
forming said curved portion between said electrode outlet opening and said proximal end opening of said cannula shaft to define said active tip portion, extending between said distal end opening and said curved portion, which has said predetermined slant angle with respect to said longitudinal axis of said cannula shaft.

14. The cannula for thermal ablation, as recited in claim 10, wherein said electrode guiding lobe is formed by:
forming an elongated longitudinal hole in form of U-shape in said short side of said active tip portion of said cannula shaft of said cannula and said electrode guiding lobe which is defined to have two lobe sides and a lobe end, wherein said two lobe sides and said lobe end are surrounded by said U shape hole;
bending said electrode guiding lobe into said inner electrode passage of said cannula about a root end thereof until said lobe end is in contact with an opposing inner surface of said inner electrode passage, such that said elongated longitudinal hole forms said electrode outlet opening of said cannula to communicate said inner electrode passage with outside and said electrode guiding lobe forms an inclined stopper for said distal end opening of said cannula to prevent any tissue entered through said distal end opening from blocking said electrode outlet opening and entering a portion of said inner electrode passage extending between said electrode guiding lobe and said proximal end opening of said inner electrode passage; and
forming said curved portion between said electrode outlet opening and said proximal end opening of said cannula shaft to define said active tip portion, extending between said distal end opening and said curved portion, which has said predetermined slant angle with respect to said longitudinal axis of said cannula shaft.

15. The cannula for thermal ablation, as recited in claim 10, wherein said electrode guiding lobe is formed by:
forming an elongated longitudinal hole in form of U-shape in said short side of said active tip portion of said cannula shaft of said cannula and said electrode guiding lobe which is defined to have two lobe sides and a lobe end, wherein said two lobe sides and said lobe end are surrounded by said U shape hole;
bending said electrode guiding lobe into said inner electrode passage of said cannula about a root end thereof until said lobe end is in contact with said opposing inner surface of said inner electrode passage, such that said elongated longitudinal hole forms said electrode outlet opening of said cannula to communicate said inner electrode passage with outside and said electrode guiding lobe forms an inclined stopper for said distal end opening of said cannula to prevent any tissue entered through said distal end opening from blocking said electrode outlet opening and entering a portion of said inner electrode passage extending between said electrode guiding lobe and said proximal end opening of said inner electrode passage; and
forming said curved portion between said electrode outlet opening and said proximal end opening of said cannula shaft to define said active tip portion, extending between said distal end opening and said curved portion, which has said predetermined slant angle with respect to said longitudinal axis of said cannula shaft.

16. A producing method of an electrode guiding arrangement of a radiofrequency cannula, comprising steps of:
forming an elongated longitudinal hole in form of U-shape in a short side of an active tip portion of an elongated and tubular cannula shaft of said radiofrequency cannula and an electrode guiding lobe having two lobe sides and a lobe end, wherein said two lobe sides and said lobe end are surrounded by said U shape hole;
bending said electrode guiding lobe into an inner electrode passage of said cannula shaft of said radiofrequency cannula about a root end thereof until said lobe end is in contact with an opposing inner surface of said inner electrode passage, such that said elongated longitudinal hole forms a radial electrode outlet opening of said radiofrequency cannula to communicate said inner electrode passage with outside and said electrode guiding lobe forms an inclined stopper for a distal end opening of said radiofrequency cannula to prevent body tissue entered through said distal end opening from blocking said electrode outlet opening and entering a portion of said inner electrode passage extending between said electrode guiding lobe and a proximal end opening of said inner electrode passage; and
forming a curved portion between said electrode outlet opening and said proximal end opening of the cannula shaft to define an active tip portion, extending between said distal end opening and said curved portion, which has a predetermined slant angle with respect to a longitudinal axis of said cannula shaft of said radiofrequency cannula.

17. The producing method, as recited in claim 16, further comprising a step of inserting a mandrel into said inner electrode passage of said radiofrequency cannula and extended from said proximate end opening to said electrode guiding lobe.

18. The producing method, as recited in claim 16, further comprising a step of forming an insulation to cover said cannula shaft of said radiofrequency cannula, wherein said insulation is extended between said proximate end opening and said curved portion of said cannula shaft.

19. The producing method, as recited in claim 16, wherein said electrode outlet opening is an elongated side port sized and shaped to allow an active electrode tip of an electrode shaft of a radiofrequency electrode, which is inserted into said inner electrode passage through said proximate end opening, being guided by said electrode guiding lobe to extend out through said electrode outlet opening so as to define a predetermined angle between said active electrode tip of said radiofrequency electrode and said active tip portion of said radiofrequency cannula.

20. The producing method, as recited in claim 18, wherein said electrode outlet opening is an elongated side port sized and shaped to allow an active electrode tip of an electrode shaft of a radiofrequency electrode, which is inserted into said inner electrode passage through said proximate end opening, being guided by said electrode guiding lobe to extend out through said electrode outlet opening so as to define a predetermined angle between said active electrode tip of said radiofrequency electrode and said active tip portion of said radiofrequency cannula.
